(19) United States
(12) Patent Application Publication
(10) Pub. No.: US 2018/0280444 A1
(43) Pub. Date: Oct. 4, 2018

(54) NANOPARTICLE BASED TUMOR ENDOTHELIAL TARGETING VACCINE

(71) Applicant: Batu Biologics, Inc., San Diego, CA (US)

(72) Inventors: Samuel C. Wagner, San Diego, CA (US); Thomas E. Ichim, San Diego, CA (US)

(73) Assignee: Batu Biologics, Inc., San Diego, CA (US)

(21) Appl. No.: 15/619,021

(22) Filed: Jun. 9, 2017

Related U.S. Application Data
(60) Provisional application No. 62/348,738, filed on Jun. 10, 2016.

(51) Int. Cl. A61K 35/44; A61K 39/00; A61K 31/235

(52) U.S. Cl. CPC A61K 35/44; A61K 39/0011; A61K 2039/572; A61K 2039/575; A61K 31/235

(57) ABSTRACT

Disclosed are methods, compositions of matter, and therapeutic protocols for treatment of cancer by selectively inducing immune responses against blood vessels feeding neoplastic tissue. In one embodiment, placental endothelial cells are stimulated with Phorbol Myristate Acetate (PMA) to induce release of nanoparticles that possess ability to: a) be taken up by antigen presenting; b) presented through direct and indirect presentation to CD4 and CD8 T cells, respectively; and c) utilized to stimulate cytoxic T cellular and humoral responses to selectively inhibit angiogenesis of tumors.

### CROSS-REFERENCE TO RELATED APPLICATION

This application claims the benefit of U.S. Provisional Application No. 62/348,738 filed on Jun. 10, 2016, entitled “NANOPARTICLE BASED TUMOR ENDOTHELIAL TARGETING VACCINE”, the contents of which are incorporated herein by reference as though set forth in their entirety.

### FIELD OF THE INVENTION

The invention pertains to the field of nanotechnology, more importantly, the invention pertains to inhibiting tumor blood vessel growth through the stimulation of immunity by nanoparticles similar to exosomes.

### BACKGROUND OF THE INVENTION

Stimulation of immunity relies on effective presentation of antigens to antigen presenting cells (APC), which in turn present antigens, after proper processing, to T cells. This process controls the quantity and quality of immune responses. Of particular importance is in the case of vaccination, where the route and presentation means of antigens control whether immune response type, specifically whether Th1, Th2, or Treg responses will ensue. One way of increasing immunogenicity of antigens is by presentation through dendritic cells. It is known that dendritic cells are the most potent APC in the body, possessing ability to uniquely activate naïve T cells. Previous experiments have demonstrated that dendritic cells are useful at breaking tolerance to cancer and stimulating productive immune responses.

Numerous animal models have demonstrated that in the context of neoplasia DCs can bind to and engulf tumour antigens that are released from tumor cells, either alive or dying, and cross-present these antigens to T cells in tumour-draining lymph nodes. This results in the generation of tumour-specific immune responses that have been demonstrated to inhibit tumor growth or in some cases induced transferrable immunological memory. Mechanistically, DCs recognize tumors using the same molecular means that they would use to recognize apoptotic cells, or cells that are stressed. One set of signals are molecules released from apoptotic cells, which are highly released by tumors, these include the nucleotides UTP and ATP, fractalkine, lipid lysophosphatidylcholine, and sphingosine 1-phosphate. Signals from stressed cells, such as tumor cells include externalization of phosphatidylserine onto the outside of the cell membrane, calreticulin, αvβ5 integrin, CD36 and lactadherin. There is some evidence that dendritic cells actively promote tumor immunity in that patients with dendritic cell infiltration of tumors generally have a better prognosis.

While DC themselves are part of the initial immune response to cancer, numerous mechanisms are used by tumors to suppress the ability of DC to stimulate an immune response. One particular mechanism is the depletion of tryptophan in the tumor microenvironment by production of indolamine 2,3 deoxygenase (IDO), which will be discussed in detail in Section 5.6. Tryptophan depletion results in T cell apoptosis, while catabolites of tryptophan depletion are known to lead to suppression of T cell activation. In order to overcome issues associated with local tumor suppression of DC, numerous studies have utilized ex vivo generated DC that have been pulsed with tumor antigen as a means of stimulating anticancer immunity.

Despite these characteristics of DC, the ability to properly distribute antigens of an inoculated vaccine to all DC or professional APC in the body is limited. In the current invention microvesicles generated from endothelial cell vaccines are utilized as a means of distributing antigen systemically throughout the body for superior antigen presentation and immune activation.

### DETAILED DESCRIPTION OF THE INVENTION

The invention disclosed provides means of generating nanoparticles from endothelial cell based vaccines for the induction of immune effector cells. Specifically, the invention teaches that nanoparticles similar to exosomes may be forced by endothelial cells that are generated to possess functional features similar to tumor endothelial cells. In one embodiment, endothelial cells are generated from placental endothelial cells as described previously by our group, specifically, Full term placentas are collected from delivery room under informed consent. Fetal membranes are manually peeled back and the villous tissue is isolated from the placental structure. Villous tissue is subsequently washed with cold saline to remove blood and scissors are used to mechanically digest the tissue, 25 grams of minced tissue is incubated with approximately 50 ml of HBSS wih 25 mM of HEPES and 0.28% collagenase, 0.25% dispase, and 0.01% DNAse at 37 Celsius. The mixture of minced placental villus tissue and digesting solution is incubated under stirring conditions for three incubation periods of 20 minutes each. Ten minutes after the first incubation period and immediately after the second and third incubation periods, the DNAse is added to make up a total concentration of DNase, by volume, of 0.01%; In the first and second incubations, the incubation flask is set at an angle, and the tissue fragments are allowed to settle for approximately 1 minute, with 35 ml of the supernantant cell suspension being collected and replaced by 38 ml (after the first digestion) or 28 ml (after the second digestion) of fresh digestion solution. After the third digestion the whole supernatant is collected; The supernatant collected from all three incubations is pooled and is poured through approximately four layers of sterile gauze and through one layer of 70 micro meter polyester mesh. The filtered solution is then centrifuged for 1000 g for 10 minutes through diluted new born calf serum, said new born calf serum diluted at a ratio of 1 volume saline to 7 volumes of new born calf serum; The pooled pellet is then resuspended in 35 ml of warm DMEM with 25 mM HEPES containing 5 mg DNase I; The suspension is then mixed with 10 ml of 90% Percoll to give a final density of 1.027 g/ml and is centrifuged at 550 g for 10 minutes with the centrifuge brake off; The pellet is then washed in HBSS and cells are incubated for 48 hours in complete DMEM media containing 100 IU of IFN-gamma per ml.

In some embodiments placental endothelial cells are purified as endothelial precursor cells by selected for cells expressing CD31 and CD133. Said selected EPC may be expanded in vitro and cultured in conditions similar to those found in tumor microenvironments. Said molecules include PGE2, TGF-beta, IL-10, and in some conditions kynurenine. Concentration and time points for culture are assessed by ability to induce expression of tumor associated molecules such as ROBO 1-18, TEM-1, CD105 and CD93.

Subsequent to generating cells that resemble tumor endothelial cells, said cells are activated to induce production of microvesicles such as exosomes. In one embodiment addition of a PKC activator is performed. Specific PKC activators include PMA. In other embodiment stimulation of microvesicles is performed by inducing apoptosis and collecting apoptotic bodies. Numerous means are known to induce apoptosis, including exposure to irradiation, non-physiological stimuli, or oxidative stress. In a preferred embodiment ValloVax cells are treated with PMA at a concentration of 5 microgram per ml for 24 hours and microvesicles with properties of exosomes are collected. Said microvesicles are collected and concentrated by means known in the art.

This invention is more specifically based on the use of chromatography separation methods for preparing membrane vesicles, particularly to separate the membrane vesicles from potential biological contaminants. More specifically, a first object of this invention resides in a method of preparing membrane vesicles from a biological sample, characterised in that it comprises at least an anion exchange chromatography treatment step of the sample.

Indeed, the applicant has now demonstrated that membrane vesicles, particularly exosomes, could be purified by anion exchange chromatography. In this way, unexpectedly, it is demonstrated in this application that exosomes are resolved in a homogeneous peak after anion exchange chromatography. This result is completely unexpected given that exosomes are complex supramolecular objects composed, among other things, of a membrane, surrounding an internal volume comprising soluble proteins. In addition, exosomes contain membrane proteins.

Therefore, a preferred object of this invention relates to a method of preparing, particularly of purifying, vesicle membranes, from a biological sample, comprising at least one anion exchange chromatography step.

In one embodiment of the invention ValloVax or other endothelial cells resembling tumor endothelial cells are inactivated and cultured with dendritic cells in vitro, said dendritic cell exosomes are then harvested and used for immunization. Generation of dendritic cells is known in the art and may be performed according to methods described and incorporated by reference, specifically methodologies have been described for DC generation, in which the DC have been used in clinical trials of the following cancers: melanoma, soft tissue sarcoma, thyroid, glioma, multiple myeloma, lymphoma, leukemia, as well as liver, lung, ovarian, and pancreatic cancer.

To apply the invention, a strong or weak, preferably strong, anion exchange may be performed. In addition, in a specific embodiment, the chromatography is performed under pressure. Thus, more specifically, it may consist of high performance liquid chromatography (HPLC).

Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE. POROS®. SEPHAROSE®, SEPHADEX®, TRISACRYL®, TSK-GEL SW OR PW®, SUPERDEX® TOYOPEARL HW and SEPHACRYL®, for example, which are suitable for the application of this invention.

Therefore, in a specific embodiment, this invention relates to a method of preparing membrane vesicles from a biological sample, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalised.

In addition, to improve the chromatographic resolution, within the scope of the invention, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, it is preferable to use high porosity gels, particularly between 10 nm and 5 .mu.m, more preferably between approximately 20 nm and approximately 2 .mu.m, even more preferably between about 100 nm and about 1 .mu.m.

For the anion exchange chromatography, the support used must be functionalised using a group capable of interacting with an anionic molecule. Generally, this group is composed of an amine which may be ternary or quaternary, which defines a weak or strong anion exchanger, respectively.

Within the scope of this invention, it is particularly advantageous to use a strong anion exchanger. In this way, according to the invention, a chromatography support as described above, functionalised with quaternary amines, is used. Therefore, according to a more specific embodiment of the invention, the anion exchange chromatography is performed on a support functionalised with a quaternary amine. Even more preferably, this support should be selected from poly(styrene-divinylbenzene), acrylamide, agarose, dextran and silica, alone or in mixtures, and functionalised with a quaternary amine.

Examples of supports functionalised with a quaternary amine include the gels SOURCEQ. MONO Q, Q SEPHAROSE®, POROS® HQ and POROS® QE, FRACTOGEL® TMAE type gels and TOYOPEARL SUPER® Q gels.

A particularly preferred support to perform the anion exchange chromatography comprises poly(styrene-divinylbenzene). An example of this type of gel which may be used within the scope of this invention is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size.

The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 .mu.l up to 10 ml or greater. In this way, the supports available have a capacity which may reach 25 mg of proteins/ml, for example. For this reason, a 100 .mu.l column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example.

In addition, to perform this invention, it is also possible to combine the anion exchange chromatography step with a gel permeation chromatography step. In this way, according to a specific embodiment of the invention, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX® 200HR (Pharmacia), TSK G6000 (TosoHaas) or SEPHACRYL® S (Pharmacia) is preferably used.

The process according to the invention may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the invention, the biological sample is a culture supernatant of membrane vesicle-producing cells.

In addition, according to a preferred embodiment of the invention, the biological sample is treated, prior to the chromatography step, to be enriched with membrane vesicles (enrichment stage). In this way, in a specific embodiment, this invention relates to a method of preparing membrane vesicles from a biological sample, characterised in that it comprises at least: b) an enrichment step, to prepare a sample enriched with membrane vesicles, and c) a step during which the sample is treated by anion exchange chromatography and/or gel permeation chromatography.

According to a preferred embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography.

Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In an other specific embodiment, the enrichment step comprises an affinity chromatography step, optionally preceded by a step of elimination of cells and/or cell debris (clarification). A preferred enrichment step according to this invention comprises (i) the elimination of cells and/or cell debris (clarification), (ii) a concentration and (iii) an affinity chromatography.

The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 .mu.m, e.g. between 0.2 and 10 .mu.m, are preferentially used. It is particularly possible to use a succession of filters with a porosity of 10 .mu.m, 1 .mu.m, 0.5 .mu.m followed by 0.22 .mu.m.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process.

The concentration step may also be performed by ultrafiltration. In fact, this ultrafiltration allows both to concentrate the supernatant and perform an initial purification of the vesicles. According to a preferred embodiment, the biological sample (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, deshydrogenases, clotting factors, interferons, lipoproteins, or also co-factors, etc. More preferably, the support used for this chromatography step is a support as used for the ion exchange chromatography, functionalised with a dye. As specific example, the dye may be selected from Blue SEPHAROSE® (Pharmacia), YELLOW 86, GREEN 5 and BROWN 10 (Sigma). The support is more preferably agarose. It should be understood that any other support and/or dye or reactive group allowing the retention (binding) of contaminants from the treated biological sample can be used in the instant invention.

In a specific embodiment of the invention, the biological sample is obtained by subjecting a membrane vesicle-producing cell culture supernatant to at least one filtration stage.

In another specific embodiment of the invention, the biological sample is obtained by subjecting a membrane vesicle-producing cell culture supernatant to at least one centrifugation step.

In a preferred embodiment of the invention, the biological sample is obtained by subjecting a membrane vesicle-producing cell culture supernatant to at least one ultrafiltration step.

In another preferred embodiment of the invention, the biological sample is obtained by subjecting a membrane vesicle-producing cell culture supernatant to at least one affinity chromatography step.

A more specific preferred membrane vesicle preparation process within the scope of this invention comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample.

In a preferred embodiment, step b) above comprises a filtration of the culture supernatant, followed by an ultrafiltration, preferably tangential.

In another preferred embodiment, step b) above comprises a clarification of the culture supernatant, followed by an affinity chromatography on dye, preferably on Blue SEPHAROSE®.

In addition, after step c), the material harvested may, if applicable, be subjected to one or more additional treatment and/or filtration stages d), particularly for sterilisation purposes. For this filtration treatment stage, filters with a diameter less than or equal to 0.3 .mu.m are preferentially used, or even more preferentially, less than or equal to 0.25 .mu.m. Such filters have a diameter of 0.22 .mu.m, for example.

After step d), the material obtained is, for example, distributed into suitable devices such as bottles, tubes, bags, syringes, etc., in a suitable storage medium. The purified vesicles obtained in this way may be stored cold, frozen or used extemporaneously.

Therefore, a specific preparation process within the scope of the invention comprises at least the following steps: c) an anion exchange chromatography and/or gel permeation chromatography treatment of the biological sample, and d) a filtration step, particularly sterilising filtration, of the material harvested after stage c).

In a first variant, the process according to the invention comprises: c) an anion exchange chromatography treatment of the biological sample, and d) a filtration step, particularly sterilising filtration, on the material harvested after step c).

In another variant, the process according to the invention comprises: c) a gel permeation chromatography treatment of the biological sample, and d) a filtration step, particularly sterilising filtration, on the material harvested after step c).

According to a third variant, the process according to the invention comprises: c) an anionic exchange treatment of the biological sample followed or preceded by gel permeation chromatography, and d) a filtration step, particularly sterilising filtration, on the material harvested after step c).

In some embodiments addition of proteins to stimulate antigen presentation and immune activation by DC derived exosomes are disclosed. Methods are known for isolation and identification a MMV fraction by employing reactants that comprise a lipophilic membrane anchoring domain and a hydrophilic target domain, such as GPI-linked proteins, to modify the MMV outer surface, or rather the vesicles' membranes, thereby providing a painting, tagging or labeling of all MMV, including exosomes, independent of their protein constitution at the outer surface of the vesicle membrane. The method is characterised therein that the hydrophilic part of the reactant which is exposed to the surrounding environment of the MMV carries a certain type of tag, such as a peptide or protein tag, which can be used to isolate the MMV it is attached to. The method is based on a reaction between a molecule referred to as the reactant, which preferably is a GPI-anchored protein, and the MMV membrane. That way it is ensured that indeed all membrane vesicles will be tagged. Because the method according to the invention is based on a general mode of action of the insertion of glycosyl-phosphatidyl-inositol-like anchored molecules into the membranes of all MMV, there is no limitation of the method as to certain types of MMV that do express specific surface molecules. By incubating the MMV, which preferably are exosomes, with a reactant which comprises a lipophilic membrane anchoring domain and a hydrophilic target domain and/or a protein or peptide tag, all the MMV are now tagged. As a result of this or as a result of the tag being attached to this hydrophilic target domain, they can easily be isolated or purified from the reaction mix. It is also a preferred embodiment wherein the tag at the reactant, whether bound to the amphiphilic membrane anchor domain, or bound to the hydrophilic target domain, has magnetic beads attached to it. Hence while incubating the MMV in the sample with that type of reactant; the magnetic beads are attached to the vesicles membranes in a one-step reaction. In another embodiment the Ex-tag facilitates the binding or attachment of the reactant to a membrane surface, such as metal chelate adsorber membrane (for example, Sartobind's metal chelate absorbers). Metal chelate absorbers represent Immobilized Metal Affinity Chromatography (IMAC) purification devices. They can simply be used in an HPLC, FPLC or operated by hand with a syringe connected via Luer Lock. These IMAC devices can be attached to the reactant via a suitable Ex-tag, such a polyhistidine, because histidine containing proteins bind to immobilized metal ions. Especially strong interactions take place with the commonly used polyhistidine (His 6-tag) with six consecutive histidine residues. The MMV can now be incubated with a reactant which carries a polyhistidine as EX-tag and is therefore attached to the membrane and can be concentrated or isolated from cell lysates or culture supernatant, by incubating and filtrating.

In one embodiment of the present invention therefore is a method for exogenously modifying the membrane composition of a MMV so as to allow for binding of proteins that increase immunogenicity such as HMGB1, comprising the steps of incubating an aqueous sample that comprises MMV with a reactant comprising a hydrophilic target domain or moiety covalently linked to an amphiphilic membrane anchor domain comprising of a lipophilic part and a hydrophilic part, wherein the lipophilic part of the membrane anchor domain becomes integrated into the lipid double layer of the membrane and wherein the hydrophilic part of the anchor domain, as well as the hydrophilic target domain become exposed to the surrounding aqueous sample fluid, and wherein the hydrophilic target domain or the hydrophilic part of the anchor domain carries an EX-tag. Such EX-tag may be a peptide a protein tag, which again may be attached to magnetic beads. Preferably the EX-tag is a protein or peptide tag. A preferred protein or peptide tag in the embodiments described below would be a Histidine-tag (His-tag), FLAG-tag, Strep-tag, FLAG-tag, GST-tag, a Myc-tag, a HA-tag or an OMP A-tag or other single or several amino acid(s) that can be chemically modified to allow attachment of a second peptide or protein tag or bioactive molecule, or a chemical entity or an organic or non-organic micro- or nano- or bead or related type of particle, characterised as enabling the isolation of the so-tagged MMV (or enveloped virus particle). A peptide tag herein is understood to comprise of at least one amino acid. The stretch of amino acids (sequence) of the peptide tag is characterised as enabling the binding to a biological, chemical or metal-based or metal-related reagent, designed for the purpose of binding to the tag and thereby allowing the isolation of the MMV. This amino acid stretch may comprise a histidine (His-) tag, a Flag-tag, a strep-tag, a one Strep-tag, a GST-tag, a Myc-tag, a HA-tag or an OMP A tag, or other amino acid(s) which enable attachment of a second peptide or protein tag. It is a preferred embodiment wherein the EX-tag is an epitope tag. The epitope tag allows the according antibody to find the protein, or in this case the anchored protein, i.e. the membrane-modified vesicle, enabling lab techniques for localization, purification, and further molecular characterization. Common epitopes used for this purpose are c-myc, HA, FLAG-tag, GST and 6× or 10× His.

1. An immunogenic composition generated by the steps of:
a) obtaining an endothelial population;
b) treating said endothelial cell population in a manner to endow a phenotype similar to endothelial cells associated with tumor blood vessels;
c) stimulating said treated endothelial cells with conditions capable of inducing microvesicle release;
d) collecting said microvesicles; and
e) administering said microvesicles in a vehicle capable of allowing for significant stimulation of immunity by said microvesicles or phagocytic cells that have uptaken said microvesicles.

2. The composition of claim 1, wherein said endothelial populations are placental endothelial cells.

3. The composition of claim 1, wherein said endothelial populations are endothelial progenitors cells.

4. The composition of claim 1, wherein said endothelial populations are immortalized endothelial cells.

5. The composition of claim 4, wherein said immortalized endothelial cells are transformed by exposure to SV40.

6. The composition of claim 4, wherein said immortalized endothelial cells are transformed by transfection with hTERT.

7. The composition of claim 4, wherein said immortalized endothelial cells are transformed by transfection with c-myc.

8. The composition of claim 4, wherein said immortalized endothelial cells are transformed by transfection with RAS.

9. The composition of claim 4, wherein said immortalized endothelial cells are transformed by transfection with CTCFL.

10. The composition of claim 1, wherein said endothelial cells are cocultured with tumor derived factors.

11. The composition of claim 1, wherein said endothelial cells are cocultured with TGF-beta.

12. The composition of claim 1, wherein said endothelial cells are cocultured with PGE-2.

13. The composition of claim 1, wherein said endothelial cells are cocultured with IL-10.

14. The composition of claim 1, wherein said endothelial cells are cocultured with kynurenine.

15. The composition of claim 1, wherein said endothelial cells are cultured under conditions capable of stimulating expression of markers associated with endothelial cells associated with tumor cells, said markers selected from a group comprisings of:
a) survivin;
b) ROBO 1-18;
c) TEM-1;
d) CD104; and
e) CD93.

16. The composition of claim 1, wherein an agent capable of stimulating HLA expression is added to said endothelial cultures to augment immunogenicity.

17. The composition of claim 16, wherein said agents capable of stimulating HLA are selected from a group comprised of:
a) interferon gamma;
b) inhibitors of CLIP;
c) toll like receptor agonists; and
d) activators of PAMPs.

18. The composition of claim 1, wherein said microvesicles are exosomes.

19. The composition of claim 1, wherein said microvesicles are apoptotic bodies.

20. The composition of claim 1, wherein immunogenic agents are chemically attached to said microvesicles.
